(19) United States
(12) Patent Application Publication
(10) Pub. No.: US 2023/0210606 A1
(43) Pub. Date: Jul. 6, 2023

(54) DETECTION OF SURGICAL TABLE MOVEMENT FOR COORDINATING MOTION WITH ROBOTIC MANIPULATORS

(71) Applicant: Asensus Surgical US, Inc., Durham, NC (US)

(72) Inventors: Kevin Andrew Hufford, Cary, NC (US); Alexander John Maret, Apex, NC (US); Matthew Robert Penny, Holly Springs, NC (US); Anthony Fernando, Chapel Hill, NC (US)

(21) Appl. No.: 18/092,191

(22) Filed: Dec. 30, 2022

Related U.S. Application Data
(60) Provisional application No. 63/295,405, filed on Dec. 30, 2021.

(51) Int. Cl. A61B 34/20; A61B 34/30

(52) U.S. Cl. CPC A61B 34/20; A61B 34/30; A61B 2034/2048; A61B 2034/2074; A61B 2034/2072; A61B 2034/301

(57) ABSTRACT

An position sensor such as an IMU is removably positioned on a patient bed used to support a patient during a robotic surgical procedure in which a robotic manipulator is used to manipulate a surgical instrument. When the bed is moved during the course of surgery, signals corresponding to a sensed changed in the bed's position are received by a processor, which causes a corresponding repositioning of the robotic manipulator.

This application claims the benefit of U.S. Provisional Application No. U.S.63/295,405, filed Dec. 20, 2021, which is incorporated herein by reference.

### BACKGROUND

During a surgical procedure, it is common for the orientation of the operating table to be adjusted for a variety of reasons: surgical site exposure, patient respiration, moving between quadrants during a procedure, etc. In robotic surgery, movement of the operating table or patient can require corresponding repositioning of the manipulators carrying the instruments. Coordinated motion between the patient/table and the manipulator arms may be desirable, but in many cases the operating room uses a patient table that is not on a commonly controlled with the manipulators. For example, the Senhance Surgical System, manufactured by Asensus Surgical, is compatible for use with a variety of patient tables, avoiding the need for a hospital to purchase a special table to be used with the surgical system. This application describes systems and methods by which operating table motion may be detected and used by the manipulator system, without requiring a direct connection between the table system and the manipulator system.

### BRIEF DESCRIPTION OF THE DRAWINGS

FIG. 1 is a perspective view of a robot-assisted surgical system on which the configurations described herein may be included;

FIG. 2 is a perspective view of a robotic manipulator arm with an instrument assembly mounted to the end effector;

FIG. 3 is a perspective view showing the end effector of the manipulator of FIG. 2, with the surgical instrument mounted to the end effector;

FIG. 4 is a perspective view similar to FIG. 3, showing the surgical instrument separated from the end effector;

FIGS. 5A-5C each show a patient bed with IMUs positioned to detect bed motion.

FIG. 6 is a block diagram schematically depicting components of an exemplary system for coordinating robotic manipulator positioning with patient bed position.

### DETAILED DESCRIPTION

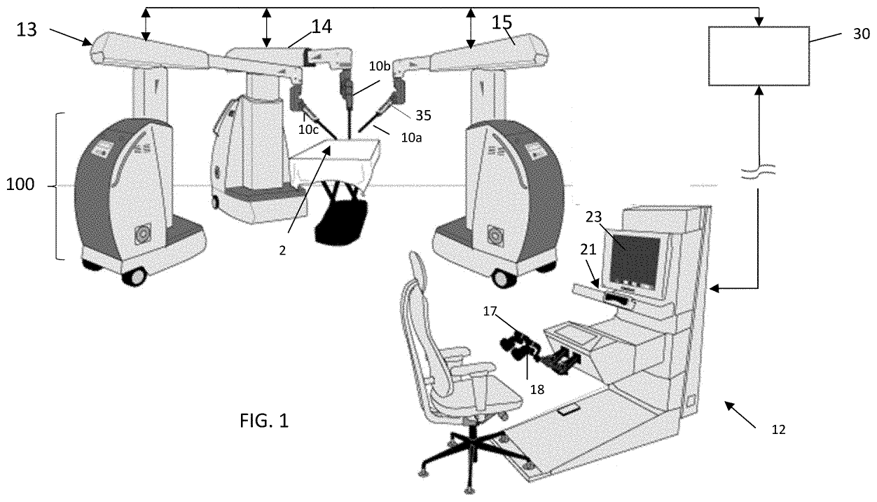

Although the inventions described herein may be used on a variety of robotic surgical systems, the embodiments will be described with reference to a system of the type shown in FIG. 1. In the illustrated system, a surgeon console 12 has two input devices such as handles 17, 18 that the surgeon selectively assigns to two of the robotic manipulators 13, 14, 15, allowing surgeon control of two of the surgical instruments 10a, 10b, and 10c disposed at the working site at any given time. To control a third one of the instruments disposed at the working site, one of the two handles 17, 18 may be operatively disengaged from one of the initial two instruments and then operatively paired with the third instrument. Or, as described below, an alternative form of input such as eye tracker 21 may generate user input for control of the third instrument. A fourth robotic manipulator, not shown in FIG. 1, may support and maneuver an additional instrument.

One of the instruments 10a, 10b, 10c is a laparoscopic camera that captures images for display on a display 23 at the surgeon console 12. The camera may be moved by its corresponding robotic manipulator using input from an eye tracker 21 or using input from one of the input devices 17, 18.

The input devices at the console may be equipped to provide the surgeon with tactile feedback so that the surgeon can feel on the input devices 17, 18 the forces exerted by the instruments on the patient's tissues.

A control unit 30 is operationally connected to the robotic arms and to the user interface. The control unit receives user input from the input devices corresponding to the desired movement of the surgical instruments, and the robotic arms are caused to manipulate the surgical instruments accordingly.

In this embodiment, each arm 13, 14, 15 is separately positionable within the operating room during surgical set up. In other words, the bases of the arms are independently moveable across the floor of the surgical room. These may be on any time of wheel, caster etc. that allow a user to easily change the position of the base on the floor of the operating room. This configuration differs from other systems that have multiple manipulator arms on a common base, and for which the relative positions of the arms can thus be kinematically determined by the system. However, although the inventive concepts described herein may be used in such systems if those systems are used together with other separately positionable components.

The patient bed 2 and the surgeon console 12, as well as other components such as the laparoscopic tower (not shown) may be likewise separately positionable.

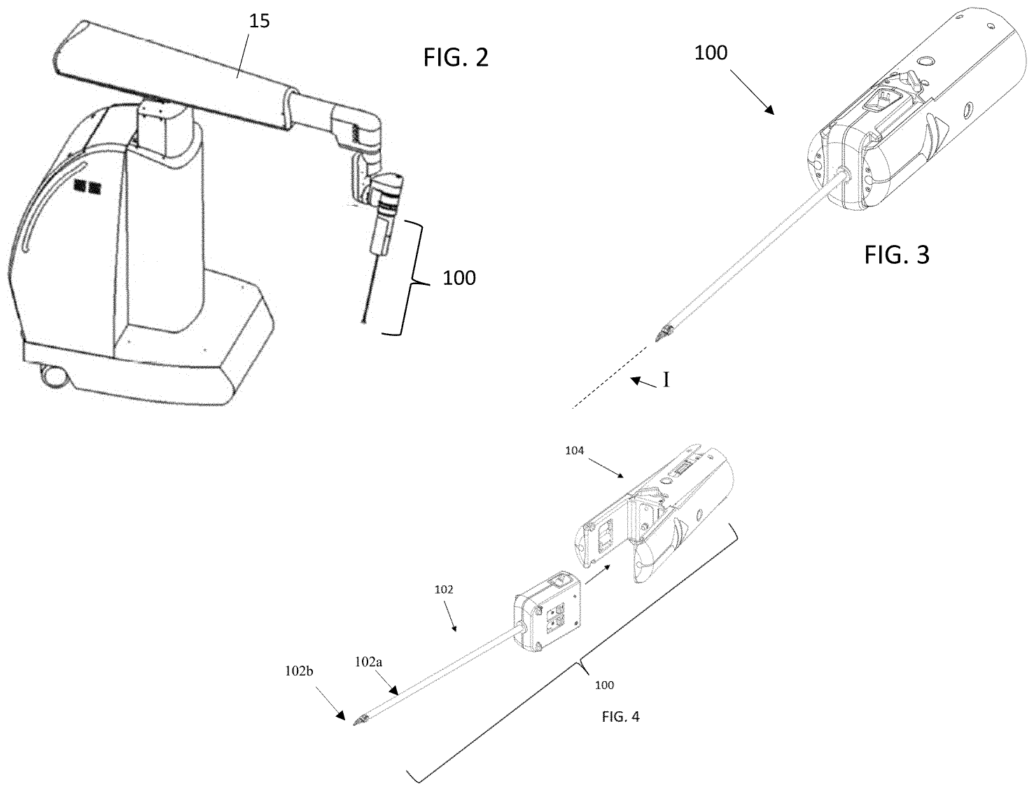

Referring to FIGS. 2-4, at the distal end of each manipulator 15 is an assembly 100 of a surgical instrument 102 and the manipulator's end effector 104. In FIGS. 3 and 4, the end effector 104 is shown separated from the manipulator for clarity, but in preferred embodiments the end effector is an integral component of the manipulator arm. The end effector 104 is configured to removably receive the instrument 102 as illustrated in FIG. 4. During a surgical procedure, the shaft 102a of the surgical instrument is positioned through an incision into a body cavity, so that the operative end 102b of the surgical instrument can be used for therapeutic and/or diagnostic purposes within the body cavity. The robotic manipulator robotically manipulates the instrument 102 in one or more degrees of freedom during a procedure. The movement preferably includes pivoting the instrument shaft 102a relative to the incision site (e.g., instrument pitch and/or yaw motion), and axially rotating the instrument about the longitudinal axis of the shaft. In some systems, this axial rotation of the instrument may be achieved by rotating the end effector 104 relative to the manipulator. Further details of the end effector may be found in commonly owned US Publication 2021/169595 entitled Compact Actuation Configuration and Expandable Instrument Receiver for Robotically Controlled Surgical Instruments, which is incorporated herein by reference. These figures show but one example of an end effector assembly 100 with which the disclosed system and method may be used, and it should be understood that the system and method are suitable for use with distinct types of end effectors.

### First Embodiment

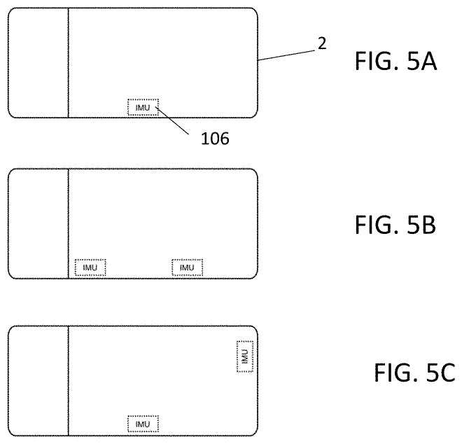

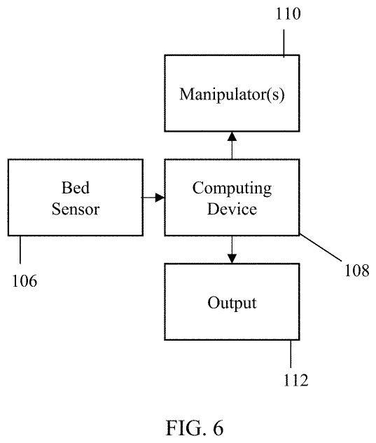

Referring to FIGS. 5A-5C, a first embodiment makes use of one or a plurality of sensors 106 to detect motion of an operating table 2 (OR table, bed, etc.). The sensors may be inertial measurement units (IMU), accelerometers, inclinometers, etc. or a combination thereof. Multiple potential implementations are illustrated in FIGS. 5A-C. In some embodiments, the rotational axis of an IMU (gyroscope) is aligned with the tilt axis of operating table. In others, multiple sensors may be placed in locations around the operating table to better differentiate between actual, intended operating table motions and other inputs (vibrations, etc.) that occur from surgical motions, collisions from other users with table, etc. Filtering (using, for example, low-pass filters, Kalman filters, etc.) of accelerometer signals and or gyroscope signals and intelligent sensor fusion are within the scope of the invention.

In preferred embodiments, the sensors are removably attached to the table rather than being integrated. This allows any surgical table to be equipped with a sensor, allowing automatic or semi-automatic motion of the robotic system manipulators in response to table motion. In alternative embodiments, the sensors are not physically attached to the table. For example, IMUs may be mounted on a patient-worn wristband, ankle band, or positioned on other equipment that is coupled to the patient, such as monitoring devices, airway devices or masks, caps, etc.

In some embodiments, table tracking is performed using alternate sensors. For example, optical markers (retroreflective IR, or IR emitters) may be positioned on the table or patient and used to provide a target for tracking via a camera or set of cameras. In still other implementations, a bed-mounted camera may sense motion relative to the room, the system, or fiducials marked on the ceiling or floor, etc. In other implementations, the drape or shape of the patient may be sensed with cameras, structured light, time-of-flight, etc.

In other implementations, a pressure-sensing mat disposed beneath the patient may sense weight shifts/pressure points and infer bed motion.

As yet another alternative, a passive physical, multi joint arm attached to a portion of the bed may include joint sensors or other sensor types that allow detection of detect bed motion relative to a base.

Referring to FIG. 5, the output from the sensor 106 that detects bed motion is received by a processor 108 associated with the robotic manipulators 110. Wired or wireless communication may be used to transmit data concerning table motion and position to the system's processor. The processor may then generate instructions that cause the manipulators to move in a manner that follows the motion of an operating table (admittance control/impedance control). Likewise, the processor may be programmed to prevent manipulator motion during periods when motion of an operating table is determined to be occurring. Still other functions may be triggered when it is detected that bed motion is occurring. For example, the processor may generate signals that cause the robotic system to release the jaws/graspers of surgical tools disposed within the patient, and/or move the manipulator arms so as to retract tools to a safe position (a position where the instrument is retracted into the trocar, for instance). Where the system controls movement of surgical instruments relative to a fulcrum determined at the incision point, the processor may cause a system alert on an output device 112 recommending that the user re-set the virtual fulcrum. The output device 112 may be a visual display (e.g. an illuminated light or LED on the manipulator or surgeon console or other structure in the room, or an alert on an image display or other display at the surgeon console 23 or elsewhere), and auditory output, a tactile output (e.g. a haptic alert on the inputs 17, 18). Alternatively or additionally, the system may cause an automatic reset of the virtual fulcrum if it has been determined that table motion has occurred and fulcrum forces exceed prior amounts or a predetermined force/torque threshold. Determination of a suitable fulcrum point for a robotic surgical manipulator is described in U.S. Pat No. 9,855,662, which is incorporated herein by reference.

All patents and applications referenced herein, including for purposes of priority, are incorporated herein by reference.

We claim:

1. A surgical method, comprising:
positioning a patient on a support;
placing a position sensor on the support;
positioning a surgical instrument on a robotic manipulator;
introducing the surgical instrument through an incision in the patient;
receiving input from a user input, and causing the robotic manipulator to manipulate the surgical instrument in accordance with the user input;
receiving signals from the position indicating a change in the position of the support; and
repositioning the robotic manipulator in response to the signals from the sensor.

2. The method of claim 1, wherein the sensor is an inertial measurement unit.

3. The method of claim 1, wherein the manipulating step includes pivoting the surgical instrument relative to a defined fulcrum, and wherein the method further includes:
in response to the signals from the sensor, re-calculating the defined fulcrum.
